(19) United States
(12) Patent Application Publication
(10) Pub. No.: US 2012/0191104 A1
(43) Pub. Date: Jul. 26, 2012

(54) Depth Probe for the Humeral Head

(76) Inventors: Reto JOST, Bern (CH); Gavin Thomas, Bellach (CH)

(21) Appl. No.: 13/011,307

(22) Filed: Jan. 21, 2011

(51) Int. Cl. A61B 17/56

(52) U.S. Cl. 606/102

(57) ABSTRACT

A method for treating fractures comprises positioning a bone fixation device along a bone and creating a first hole from an outer surface of the bone through a lateral cortex to a central portion thereof, the first hole corresponding to a position at which a first bone fixation element receiving opening is to be located when the bone fixation device is coupled to the bone in a desired orientation in combination with inserting and pushing a probe through the central portion of the bone until a tip of the probe contacts an interior structure of the bone providing resistance to penetration by the probe increased relative to the central portion of the bone and determining a first length of the portion of the probe extending into the bone via the first hole to select a desired length of a first bone fixation element to be inserted therethrough.

### BACKGROUND

Proximal humeral fractures are common in osteoporotic bone and may be fixed using a bone plate and a plurality of bone screws. For optimal fixation, the screw tip should be in close proximity to the far cortex. Determining a length of bone screw that should be used for fixing the plate to the humeral head, however, is a difficult process since the far cortex of the humeral head is often thin and weak. Currently, the length of screw is determined by pre-drilling a hole into the humeral head and inserting a standard depth gauge therein to measure a depth of the pre-drilled hole. The depth gauge in this process, however, is not guided so that the measured depth may be inaccurate, resulting in the use of an incorrect screw length. Studies have shown that the rate of screw tips penetrating into the joint is as high as 14%.

### SUMMARY OF THE INVENTION

The present invention is directed to a method for treating a fractures comprising positioning a bone fixation device along a bone, the bone fixation device including a first bone fixation element receiving opening and creating a first hole from an outer surface of the bone through a lateral cortex thereof to a central portion of the bone, the first hole corresponding to a position at which the first bone fixation element receiving opening is to be located when the bone fixation device is coupled to the bone in a desired orientation in combination with inserting a probe through the first hole and pushing the probe through the central portion of the bone until a tip of the probe contacts an interior structure of the bone providing resistance to penetration by the probe increased relative to the central portion of the bone and determining a first length of the portion of the probe extending into the bone via the first hole. A length of a length of a first bone fixation element to be inserted into the bone via the first bone fixation opening is then selected based on the first length of the portion of the probe extending into the bone via the first hole.

The present invention is further directed to a system for treating bone comprising a bone fixation device extending from a proximal end to a distal end and including a first bone fixation receiving opening extending therethrough and a drill including a drill bit configured to drill a first hole from an outer surface of the bone through a lateral cortex thereof in a position at which the first bone fixation element receiving opening is to be located when the bone fixation device is coupled to the bone in a desired orientation in combination with a probe sized and shaped to be inserted through the first hole and into the bone, the probe including a blunt tip providing tactile feedback upon contact with interior structures of the bone and markings formed along a length of the probe indicating a first length of the probe from the blunt tip thereof. A first bone fixation element to be inserted into the bone via the first bone fixation opening has a first length selected to correspond to a length of a portion of the probe extending into the bone via the first hole.

### BRIEF DESCRIPTION OF THE DRAWINGS

FIG. 1 shows a perspective view of a system according to an exemplary embodiment of the present invention;

FIG. 2 shows a top view of an exemplary bone plate according to the system of FIG. 1;

FIG. 3 shows a side view of the bone plate of FIG. 2;

FIG. 4 shows a perspective view of an exemplary aiming device and a bone plate according to the system of FIG. 1;

FIG. 5 shows a perspective view of the aiming device of FIG. 4;

FIG. 6 shows a side view of an exemplary guide sleeve according to the system of FIG. 1;

FIG. 7 shows a side view an exemplary drill tool according to the system of FIG.1;

FIG. 8 shows a side view of an exemplary probe according to the system of FIG. 1;

FIG. 9 shows a perspective view of the guide sleeve inserted into an opening of the aiming device of the system of FIG. 1 according to an exemplary method of the present invention;

FIG. 10 shows a perspective view of the drill tool inserted through the guide sleeve according to the method of FIG. 9;

FIG. 11 shows a perspective view of the probe inserted through the guide sleeve according to the method of FIG. 9;

FIG. 12 shows a perspective view of a bone fixation element inserted through the guide sleeve according to the method of FIG. 9;

FIG. 13 shows a perspective view of the bone plate fixed to a bone via bone fixation elements according to the method of FIG. 9;

FIG. 14 shows a top plan view of the bone plate and bone fixation elements of FIG. 13; and

FIG. 15 shows a perspective view of a system according to another exemplary embodiment of the present invention.

### DETAILED DESCRIPTION

The present invention may be further understood with reference to the following description and the appended drawings, wherein like elements are referred to with the same reference numerals. The present invention relates to the treatment of bone fractures and, in particular, relates to a system and method for determining a length of a bone fixation element to be inserted into a head portion of a bone. Exemplary embodiments of the present invention describe a method of inserting a depth probe into the humeral head in a guided fashion to measure a length of screw that should be inserted therein. Although the exemplary embodiments specifically describe measuring a depth of a humeral head, it will be understood by those of skill in the art that the system and method of the present invention may be used to measure the depth of a head portion of any long bone.

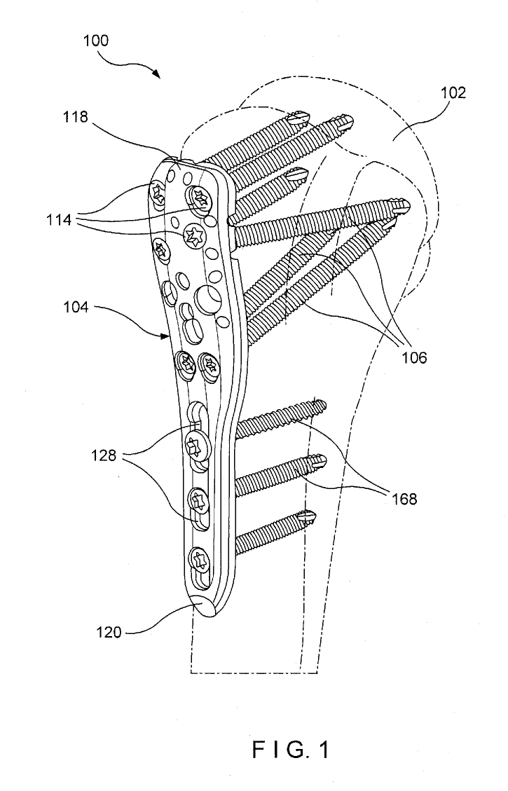

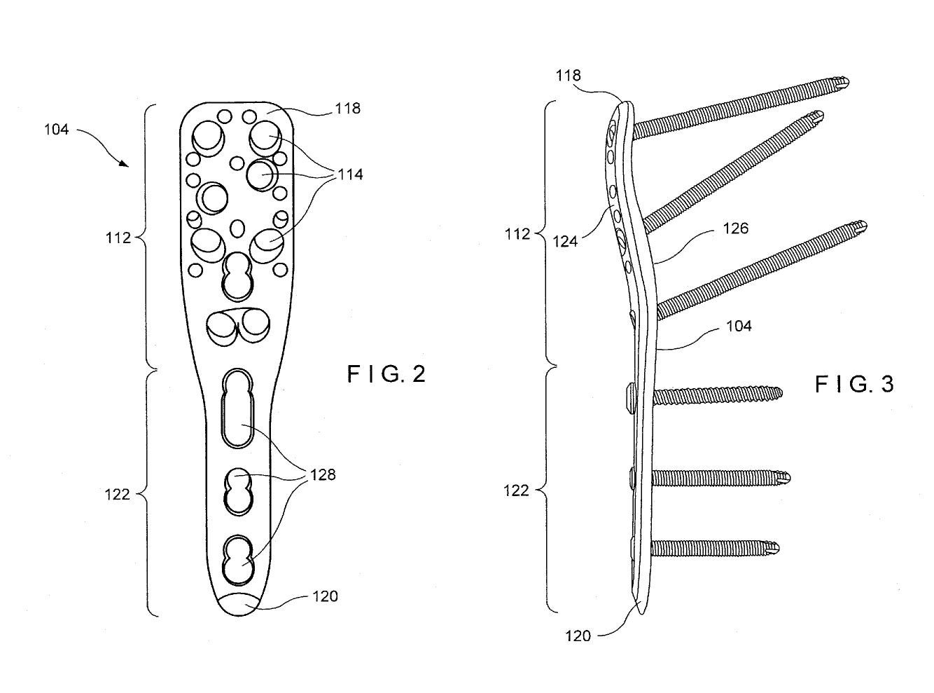

As shown in FIGS. 1-6, a system 100 for fixing a fracture according to an exemplary embodiment comprises a bone plate 104 which may be fixed to a bone 102 using a plurality of bone fixation elements 106. The bone plate 104 may be sized and shaped to be positioned along a proximal humerus such that a proximal portion 112 of the bone plate 104 lies over a humeral head. Thus, a bone fixation element 106 may pass through a proximal opening 114 in the proximal portion 112 and into the head portion of the bone 102. The system 100 further comprises an aiming device 108 and a guide sleeve 138 which may be used to guide a drill tool 110 and/or a probe 116 through the proximal opening 114 for measuring a depth of the head portion of the bone 102 so that a desired length for the bone fixation element 106 may be properly determined. Where the far cortex of the head portion of the bone 102 is thin and weak, there is a risk for the bone fixation element 106 to penetrate the far cortex of the bone 102 and extend into the joint. Thus, it is important for a surgeon or other user to accurately determine a length of the bone fixation element 106 that should be inserted into the proximal opening 114. The drill tool 110 may be used to drill a hole through the lateral cortex of the head portion of the bone 102 to permit insertion of the probe 116 therethrough. The probe 116, guided by the guide sleeve 138, may then be inserted through the drilled hole to measure a depth of the head portion of the bone 102 so that a surgeon or other user may select a bone fixation element 106 having a length corresponding to the measured depth of the head portion of the bone 102.

As shown in FIGS. 1-3, the plate 104 extends longitudinally from a proximal end 118 to a distal end 120 such that the proximal portion 112 may be positioned over the head portion of the bone 102 while a distal portion 122 extends distally therefrom to lie over a portion of a length of the bone 102. The plate 104 has a first surface 124 which, when the plate 104 is in an operative position along the bone 102, faces away from the bone 102 and a second surface 126 which, when the plate 104 is in the operative position faces the bone 102. The bone plate 104 includes a plurality of proximal openings 114 extending therethrough from the first surface 124 to the second surface 126 along the proximal portion 112 and distal openings 128 extending therethrough from the first surface 124 to the second surface 126 along a distal portion 122 of the bone plate 104. Each of the proximal openings 114 defines a central axis extending through a center thereof and is sized and shaped to receive a bone fixation element 106 therethrough along its respective central axis. The distal openings 128 may be any of a variety of types of bone fixation receiving openings such as, for example, locking holes, tapered holes, compression holes, variable angle holes or combination holes or any combination of these types of holes. That is, as would be understood by those skilled in the art, to bone plate 104 may include more than one type of distal opening 128 configured to receive different types of distal fixation elements 168 (e.g., locking screw, cortex screw) therethrough.

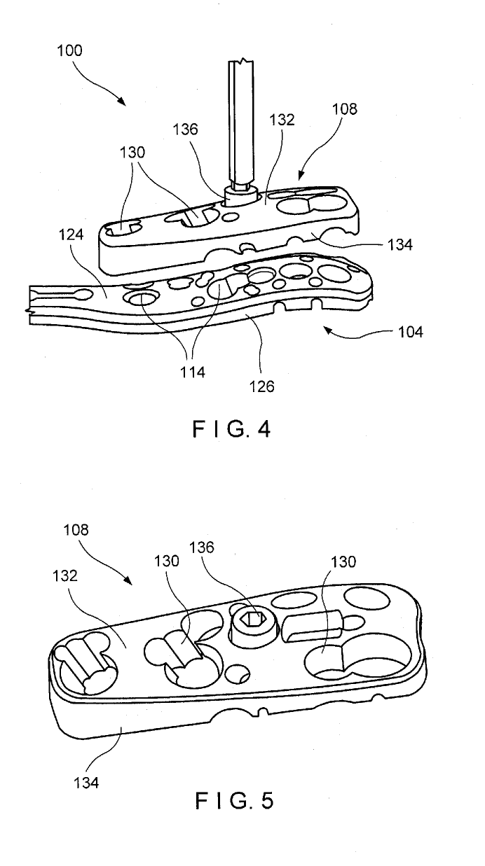

As shown in FIGS. 4-5, the aiming device 108 may be used to guide the drill tool 110 and the probe 116 through the proximal openings 114 along central axes thereof. A second surface 134 of the aiming device 108 is preferably sized and shaped to correspond to a size and contour of the first surface 124 of the proximal portion 112 of the bone plate 104 and may be configured to releasably coupled to the first surface 124 of the bone plate 104. The aiming device 108 may be coupled to the bone plate 104 via, for example, an attaching screw 136. For example, the attaching screw 136 may engage a hole in the aiming device 108 and a corresponding hole in the bone plate 104. The aiming device 108 includes a plurality of aiming holes 130 extending therethrough from a first surface 132 which, when the aiming device 108 and the bone plate 104 are coupled, faces away from the bone plate 104, to the second surface 134 which, when the aiming device 108 and the bone plate 104 are coupled, faces the bone plate 104. In the coupled configuration, each of the aiming holes 130 corresponds to and aligns with a corresponding one of the proximal openings 114. Thus, when the aiming device 108 and the bone plate 104 are coupled to one another, the central axes of the aiming holes 130 align with the central axes of the proximal openings 114. As shown in FIG. 5, the aiming holes 130 may overlap one another so long as the central axes of the aiming holes 130 are co-axial with the central axes of the proximal openings 114. Each of the aiming holes 130 is sized and shaped to receive the guide sleeve 138 therethrough.

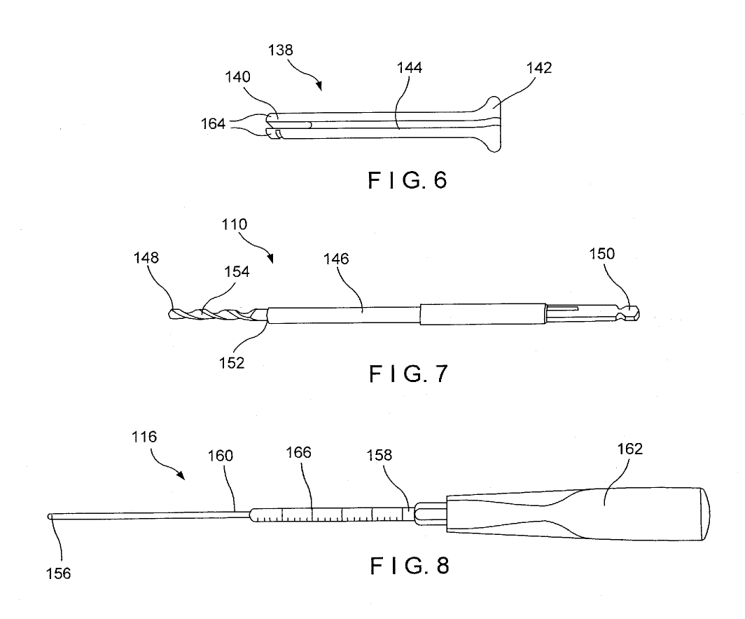

The guide sleeve 138, as shown in FIG. 6, may be substantially tubular extending longitudinally from a first end 140 to a second end 142 and including a lumen 144 extending therethrough. The lumen 144 is sized and shaped to permit the drill 110 and/or the probe 116 to be received therein and guided into the bone 102 along the central axis of one of the proximal openings 114. The first end 140 may be configured to engage an inner surface of the aiming hole 130. For example, the first end 140 may include wings 164 separated from one another via a longitudinal slot and biased toward an open configuration in which an outer diameter of the first end 140 is greater than an inner diameter of the aiming holes 130. Thus, when the first end 140 is inserted into an aiming hole 130, the wings 164 are flexed toward one another providing a friction fit with the inner surface of the aiming hole 130.

As shown in FIG. 7, the drill tool 110 includes a drill bit 146 extending longitudinally from a first drilling end 148 including, for example, cutting flutes 155, to a second end 150 configured for attachment to a drill (not shown). The drill bit 146 may have an outer diameter only slightly smaller than an inner diameter of the guide sleeve 138 so that the drill bit 146 is slidably guided therethrough. The drill bit 146 may also include a stop 152 along a length thereof to prevent the user from drilling beyond a predetermined depth (i.e., corresponding to a depth of an inner surface of a lateral cortex of the bone 102 on a side opposite the point of entry into the bone). The stop 152 may be configured as a shoulder at which a diameter of the drill bit 146 increases so that a diameter of a portion of the drill bit 146 extending from the stop 152 toward the first end 148 is smaller than a diameter of the drill bit 146 extending from the stop 152 toward the second end 150.

As shown in FIG. 8, the probe 116 includes a shaft portion 160 extending longitudinally from a first end 156 to a second end 158 attached to a handle 162 for moving the shaft portion 160 through the guide sleeve 138 and into the bone 102. The first end 156 may include a blunt tip preventing damage to the bone and providing tactile feedback to the user regarding bone density. An outer diameter of the shaft portion 160 may be sized and shaped to be slidably accommodated within the lumen 144 of the guide sleeve 138. The shaft portion 160 also includes markings 166 along a portion of a length thereof, indicating a distance from the first end 156. Thus, as the probe 116 is inserted into the head portion of the bone 102, the markings 166 indicate a length of the probe 116 inserted into the bone 102 and thus correspond to a depth through the head portion of the bone 102 to the cortex on the far side of the head portion. Thus, the depth measured via the markings 166 may be used to select a length of the bone fixation element 106 which will provide maximum purchase in the bone 102 without penetrating the cortex on the far side of the bone. Although the probe 116 is described as including markings 166 therealong, in another embodiment, the probe 116 may include other features for indicating a length of the probe 116 inserted into the bone 102. For example, the probe 116 may include a display (e.g., digital) for showing the length of the probe 116 inserted into bone. In an alternative embodiment, the markings 166 may be similarly incorporated in the guide sleeve 138 so that the length of the probe 116 inserted into the bone may be determined via markings on the guide sleeve 138.

FIGS. 9-14 illustrate a technique by which a user (e.g., surgeon) may fix a fracture of the bone 102 using the bone plate 104. The bone plate 104 is coupled to the aiming device 108 to ensure that the bone fixation elements 106 are properly inserted along the central axes of the proximal openings 114. As described above, the aiming device 108 may be releasably coupled to the bone plate 104 via, for example, the attachment screw 136. The fracture of the bone 102 is reduced and the bone plate 104, and the attached aiming device 108 are positioned along the bone 102 (e.g., along the proximal humerus) and temporarily fixed thereto via a distal fixation element 168 inserted through the distal opening 128 in the distal portion 122 of the bone plate 104. The guide sleeve 138 is then inserted into one of the aiming holes 130, as shown in FIG. 9, so that a central axis of the lumen 1144 is co-axial with the central axis of the corresponding proximal opening 114.

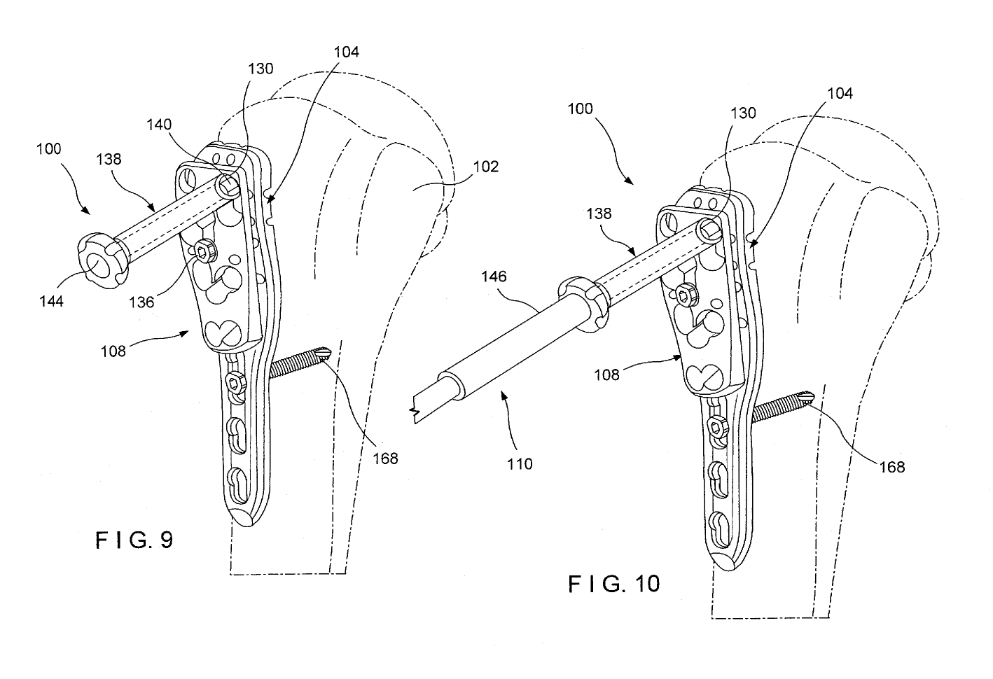

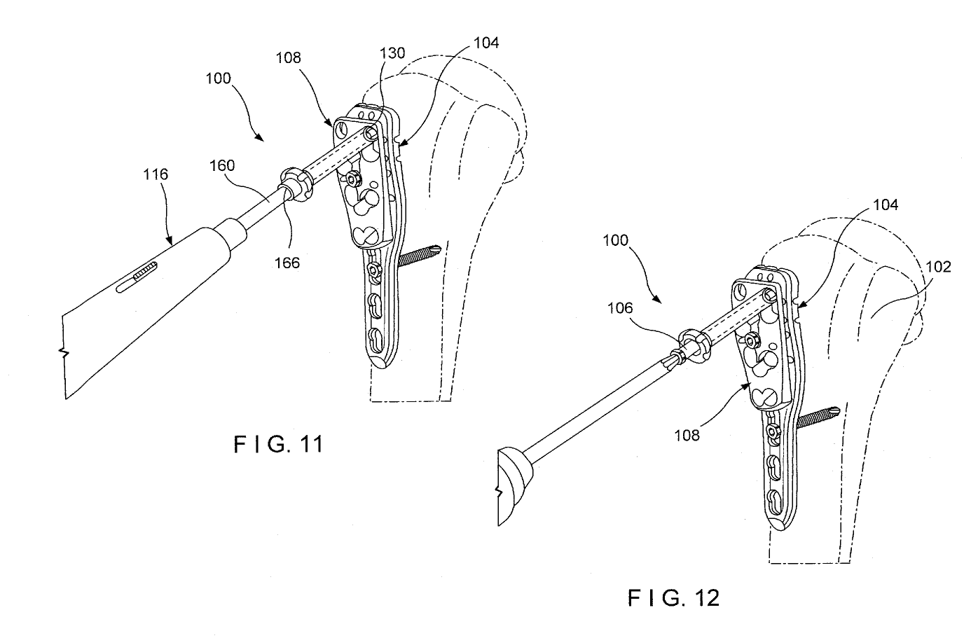

Once the guide sleeve 138 has been properly positioned in the aiming hole 130, a hole may be formed through the lateral cortex of the bone 102 using, for example, the drill bit 146 of the drill tool 110. The drill but 146 may be inserted through the lumen 144, as shown in FIG. 10, to drill through the lateral cortex of the bone 102 until the stop 152 abuts the bone 102 preventing the drill tool 110 from passing further into the bone once it has penetrated the lateral cortex on the near side of the bone 102. The drill bit 146 is removed from the guide sleeve 138 so that the shaft 160 of the probe 116 may be inserted into the lumen 144, as shown in FIG. 11, and carefully pushed through the humeral head of the bone 102 using the handle 162. Guided by the guide sleeve 138, the user continues to push the probe 116 through the bone 102 until the user feels via the blunt tip at the first end 156 the increased density indicating that the tip 156 of the probe 116 has contacted the far cortex. Once the far cortex has been detected, the user reads the markings 166 to determine the depth of the far cortex and uses this depth to select a bone fixation element 106 having a desired length.

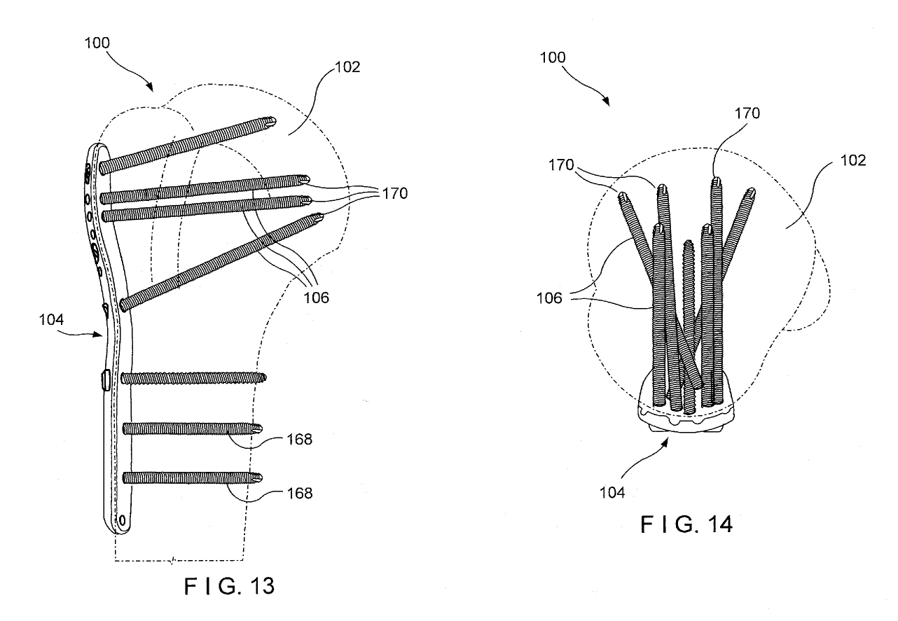

The selected bone fixation element 106 may then be inserted through the guide sleeve 138, as shown in FIG. 12, into the proximal opening 114 and driven into the bone 102 using a driving tool, as will be understood by those of skill in the art. The steps described above may be repeated for each of the proximal openings 114 with a desired length of bone fixation element 106 selected for each of the proximal openings 114. As shown in FIGS. 13-14, the bone fixation elements 106 should extend through the humeral head of the bone 102 so that the tips 170 of the bone fixation elements 106 are adjacent to the far cortex without penetrating the far cortex and, more importantly, to prevent any bone fixation element 106 from being inadvertently driven through the far cortex to project from the bone 102. Once a desired length of bone fixation element 106 has been selected for each of the proximal openings 114 and the selected bone fixation elements 106 have been inserted therethrough, the aiming device 108 and the guide sleeve 138 are de-coupled from the bone plate 104. It will be understood by those of skill in the art that once the fracture has been reduced as desired, additional bone fixation elements may be inserted through any of the remaining distal openings 128 to provide additional fixation.

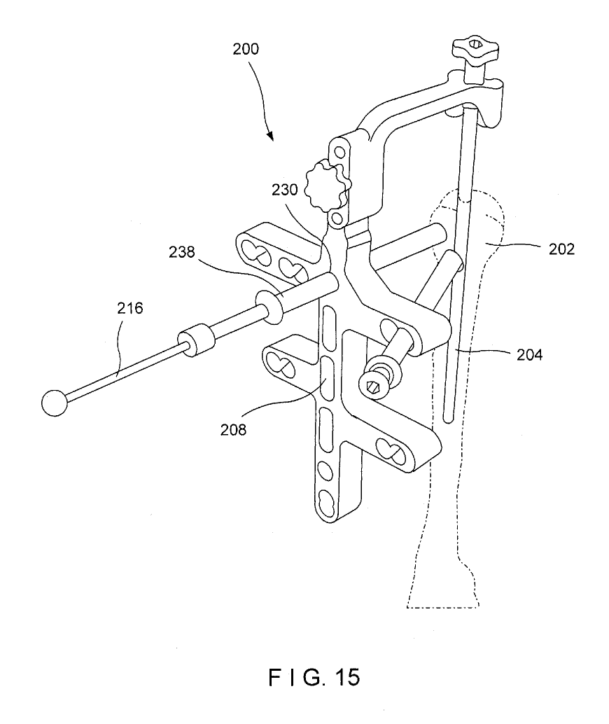

Although the method of the system 100 is specifically described with respect to the bone plate 104, it is respectfully submitted that it would be understood by those of skill in the art that the method of the present invention may be used to fix a bone fracture using any of a variety of bone fixation devices. For example, as shown in FIG. 15, a system 200 may be used to fix a fracture of a bone 202 using an intramedullary nail or rod 204, an aiming device 208, a probe 216 and a guide sleeve 238. The system 200 may be substantially similar to the system 100 and may be utilized in a substantially similar manner. The intramedullary rod 204 includes at least one opening for receiving a bone fixation element therethrough, the opening positioned within the head portion of the bone 202 when the intramedullary rod 204 is inserted into bone 202. Thus, a user may select a length of the bone fixation element by measuring a depth of the head portion through the opening. The probe 216 and the guide sleeve 238 are substantially similar to the probe 116 and guide sleeve 138 of the system 100, as described above.

The intramedullary rod 204 is inserted through the medullary canal of the bone 202 and coupled to the aiming device 208 such that an aiming hole 230 of the aiming device 208 corresponds to and aligns with the opening of the intramedullary rod 204. The guide sleeve 238, which may be substantially similar to the guide sleeve 138 described above, is inserted through the aiming hole 230 so that a lumen of the guide sleeve 238 is coaxial with a central axis of the opening of the intramedullary rod 204. Thus, as described above with respect to the system 100, a hole may be formed through only the lateral cortex of the bone 202 using, for example, a drill, such that the probe 216 may be inserted therethrough, as described above with respect to the system 100, to measure the depth of the far cortex head portion of the bone 202. The measured depth may then be used to select a desired length of a bone fixation element to be inserted through the opening of the intramedullary rod 204.

It will be understood by those skilled in the art that various modifications and variations can be made in the structure and methodology of the present invention, without departing from the spirit or scope of the invention. Thus, it is intended that the present invention cover the modifications and the variations of this invention provided that they come within the scope of the appended claims and their equivalents.

What is claimed is:

1. A method for treating a fracture, comprising:
positioning a bone fixation device along a bone, the bone fixation device including a first bone fixation element receiving opening;
creating a first hole from an outer surface of the bone through a lateral cortex thereof to a central portion of the bone, the first hole corresponding to a position at which the first bone fixation element receiving opening is to be located when the bone fixation device is coupled to the bone in a desired orientation;
inserting a probe through the first hole and pushing the probe through the central portion of the bone until a tip of the probe contacts an interior structure of the bone providing resistance to penetration by the probe increased relative to the central portion of the bone;
determining a first length of the portion of the probe extending into the bone via the first hole; and
selecting a length of a first bone fixation element to be inserted into the bone via the first bone fixation opening based on the first length of the portion of the probe extending into the bone via the first hole.

2. The method of claim 1, further comprising:
forming a second hole through the lateral cortex thereof to a central portion of the bone, the second hole corresponding to a position at which a second bone fixation element receiving opening is to be located when the bone plate is coupled to the bone in a desired orientation;
inserting a probe through the second hole and pushing the probe through the central portion of the bone until a tip of the probe contacts an interior structure of the bone providing resistance to penetration by the probe increased relative to the central portion of the bone;
determining a second length of the portion of the probe extending into the bone via the second hole; and
selecting a length of a second bone fixation element to be inserted into the bone via the second bone fixation opening based on the second length of the portion of the probe extending into the bone via the second hole.

3. The method of claim 2, further comprising inserting the first bone fixation element into the first bone fixation element receiving hole to fix the bone fixation device to the bone.

4. The method of claim 1, further comprising coupling an aiming device to the bone fixation device, the aiming device including a first aiming hole which, when the aiming device is coupled to the bone fixation device in an operative position, is coaxial with the first bone fixation element receiving opening.

5. The method of claim 4, further comprising inserting a first end of a guide sleeve into the first aiming hole so that a lumen of the guide sleeve is coaxial with the first aiming hole and the first bone fixation element receiving opening.

6. The method of claim 5, wherein the first end of the guide sleeve includes wings formed via a longitudinal slot extending along a portion of a length thereof, the wings being biased toward open configuration in which the wings are flexed apart from one another so that a diameter of a distal end of the sleeve is greater than a diameter of the first aiming hole so that, when inserted into the first aiming hole, the wings are flexed radially inward away from the open configuration to create a friction fit between the guide sleeve and the first aiming hole.

7. The method of claim 5, wherein one of the drill, the probe and the first bone fixation element is inserted into the bone via the guide sleeve.

8. The method of claim 1, wherein the first hole is formed through the lateral cortex using a drill.

9. The method of claim 8, wherein the drill includes a stop along a length thereof positioned to preventing the hole from being drilled beyond an estimated depth of the lateral cortex.

10. The method of claim 1, further comprising temporarily fixing the bone fixation device to the bone by inserting a bone fixation element through a distal opening extending through a distal portion of the bone fixation device.

11. The method of claim 1, wherein the bone fixation device is one of an intramedullary nail and a bone plate.

12. The method of claim 1, wherein the first bone fixation element extends through a proximal portion of the bone fixation device sized and shaped to be positioned over a head portion of the bone.

13. A system for treating a bone, comprising:
a bone fixation device extending from a proximal end to a distal end and including a first bone fixation receiving opening extending therethrough;
a drill including a drill bit configured to drill a first hole from an outer surface of the bone through a lateral cortex thereof in a position at which the first bone fixation element receiving opening is to be located when the bone fixation device is coupled to the bone in a desired orientation;
a probe sized and shaped to be inserted through the first hole and into the bone, the probe including a blunt tip providing tactile feedback upon contact with interior structures of the bone;
markings formed along a length of the probe indicating a first length of the probe from the blunt tip thereof; and
a first bone fixation element to be inserted into the bone via the first bone fixation opening, a first length of the first bone fixation element selected to correspond to a length of a portion of the probe extending into the bone via the first hole.

14. The system of claim 13, wherein the bone fixation device includes a second bone fixation element receiving opening.

15. The system of claim 14, further comprising a second bone fixation element to be inserted into the bone via the second bone fixation element opening and having a length selected to correspond to a second length of a portion of the probe extending into the bone via a second hole.

16. The system of claim 13, further comprising an aiming device releasably couplable to the bone fixation device, the aiming device including a first aiming hole which, when the aiming device is coupled to the bone fixation device in an operative position, is coaxial with the bone fixation element receiving opening.

17. The system of claim 16, further comprising a guide sleeve including a first end sized and shaped to be inserted into the first aiming hole so that a lumen of the guide sleeve is coaxial with the first aiming hole and the first bone fixation element receiving opening.

18. The system of claim 17, wherein the first end of the guide sleeve is separated by a longitudinal slot into lateral wings extending along a portion of a length of the guide sleeve, the wings being biased toward an open configuration in which the wings are separated from one another so that a diameter of a distal end of the sleeve is greater than a diameter of the first aiming hole so that, when inserted into the first aiming hole, the wings are flexed radially inward away from the open configuration to create a friction fit between the guide sleeve and the first aiming hole.

19. The system of claim 17, wherein one of the drill, the probe and the first bone fixation element is sized and shaped to be inserted into the bone via the guide sleeve.

20. The system of claim 13, wherein the drill includes a stop along a length thereof positioned to prevent the hole from being drilled beyond an estimated depth of the lateral cortex.

21. The system of claim 13, wherein the first bone fixation element receiving opening extends through a proximal portion of the bone fixation device and the bone fixation device includes a third bone fixation element receiving opening extending through a distal portion thereof, the third bone fixation element receiving opening sized and shaped to receive a third bone fixation element to temporarily fix the bone fixation device to the bone.

22. The system of claim 13, wherein the bone fixation device is one of an intramedullary nail and a bone plate.

23. The system of claim 13, wherein the first bone fixation element receiving opening extends through a proximal portion of the bone fixation device sized and shaped to be positioned over a head portion of the bone.

24. A method for treating a fracture, comprising:
positioning a bone plate along a bone such that a proximal portion of the plate is positioned over a head portion of the bone, the proximal portion including a bone fixation element receiving opening;
drilling a hole from an outer surface of the bone through a lateral cortex thereof to a central portion of the bone using a drill, the hole corresponding to a position at which the bone fixation element receiving opening is to be located when the bone plate is coupled to the bone in a desired orientation;
inserting a probe through the hole and pushing the probe through the central portion of the bone until a tip of the probe contacts an interior structure of the bone providing resistance to penetration by the probe increased relative to that provided by the central portion of the bone;
determining a length of the portion of the probe extending into the bone via the first hole; and
selecting a length of a bone fixation element to be inserted into the bone via the bone fixation opening based on the length of the portion of the probe extending into the bone via the hole.
